(19) United States
(12) Patent Application Publication
(10) Pub. No.: US 2019/0350769 A1
(43) Pub. Date: Nov. 21, 2019

(54) Sanitary pad

(71) Applicant: Ingrim Roberson-Mitchem, Houston, TX (US)

(72) Inventor: Ingrim Roberson-Mitchem, Houston, TX (US)

(21) Appl. No.: 15/932,899

(22) Filed: May 18, 2018

(51) Int. Cl. A61F 13/472; A61F 13/475; A61F 13/53

(52) U.S. Cl. CPC A61F 13/47236; A61F 13/15203; A61F 13/53; A61F 13/475

(57) ABSTRACT

A sanitary pad 10 is provided for eliminating incidents of leakage from the front and rear of a user's torso. The sanitary pad 10 includes a lower fluid-impervious base member 12 having engaging members 14 coupled to each side thereof for securing the pad to an under garment of a user. An intermediate liquid absorbent member 16 is adjacently coupled to the base member. A pair of spaced raised longitudinally extending absorbent guide members 20 are coupled to the intermediate absorbent member 16. A first oval shaped liquid absorbent member 22 is coupled to one end 24 and sides 26 of the intermediate absorbent member 16 and a second oval shaped liquid absorbent member 28 is coupled to another end 30 and sides 32 thereof thereby preventing leakage from the front and rear of the sanitary pad 10.

### TECHNICAL FIELD

This invention relates to a sanitary pad and more particularly to an extra absorbent sanitary pad for protecting a user during initial periods of heavy flow during the menstrual cycle. Heavy flow during the menstrual cycle can result in some anxiety for a woman because of the consequences of leakage. Obviously leakage can result from flow from front, side and rear portions of the sanitary pad. The most critical problems exist in the front and rear of the pads. One way to address that concern is by using more than one sanitary pad. This solution however does not necessarily solve the problems associated with leakage at the front and rear of the pads. Thus it is desirable to have a single sanitary pad that contains and eliminates leakage that may result from the front and rear of the pads during times of unusually heavy menstrual flow.

### BACKGROUND ART

There have been some attempts to deal with issue of leakage associated with sanitary pads. However none of these products specifically address the need to assure that there is no leakage at the front and rear of the pad. However some of the products do address the leakage issue at the rear of the pad. One such arrangement is disclosed in U.S. Pat. No. 7,196,241B2. This patent discloses a sanitary napkin in which diffusing liquid hardly reaches a rear edge that is adapted to make a liquid absorbent layer less deformable in a rear region so that it can easily be kept in close contact with the wearer's buttocks. The sanitary napkin has an elongated main absorbent region surrounded by compressed grooves that are formed in a skin-side surface. Since a rear end of the main absorbent region is far away from a rear edge of the napkin to provide a relatively large region, therefore worries about rearward leakage would be relieved.

Despite this there is still a need for a product that addresses the leakage issue at both ends of the pad during periods of heavier than usual menstrual flow.

### DISCLOSURE OF THE INVENTION

A sanitary pad is provided for eliminating incidents of leakage for a user. The sanitary pad of this invention includes a lower fluid-impervious base member. An engaging member is coupled to each side of the base member for securely engaging adjacent outside surfaces of and under garment of a user. An intermediate liquid absorbent member is adjacently coupled to the base member. The invention also includes a pair of spaced raised, longitudinally extending absorbent guide members aligned in and coupled to the intermediate member. The sanitary pad also includes a first oval shaped liquid absorbent member which is coupled to one end and sides of the intermediate liquid absorbent member and a second oval shaped liquid absorbent member which is coupled to another end and other sides of the liquid intermediate absorbent member thereby preventing leakage from the front and rear of the sanitary pad.

### BRIEF DESCRIPTION OF THE DRAWING

The details of the invention will be described in connection with the accompanying drawing in which:

FIG. 1 is a first perspective view illustrating a sanitary pad in accordance with the principles of the invention.

FIG. 2 is a second perspective view illustrating a sanitary pad in accordance with the principles of the invention.

FIG. 3 is a side cross-sectional view of the sanitary pad in accordance with the principles of the invention.

### BEST MODE FOR CARRYING OUT THE INVENTION

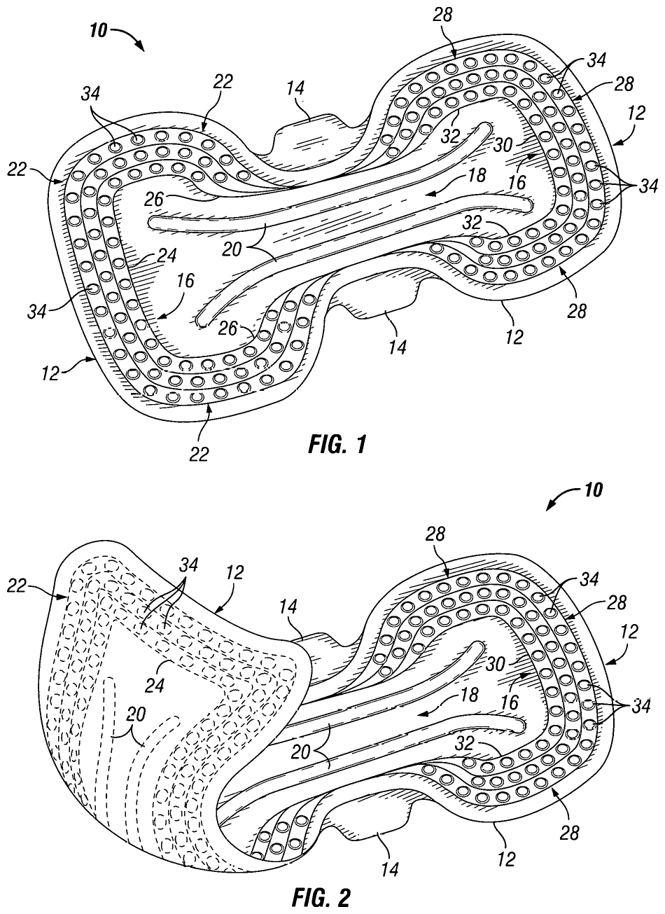

Referring to FIGS. 1, 2 and 2 there is shown, a sanitary pad, generally designated, by the numeral, 10. The sanitary pad 10 includes a lower fluid impervious base member, generally designated, by the numeral 12 (FIG. 2). The fluid-impervious base member 12, can be made, for example, of a thin plastic like material which will inhibit liquid substances from flowing therethrough. The base member 12 has formed on each side thereof a wing shaped engaging member 14 for securely engaging adjacent outside surfaces of an under garment of a user (not shown). The wing shaped engaging member 14, may be made, for example, with an adhesive agent to enhance coupling engagement with the under garment of the user.

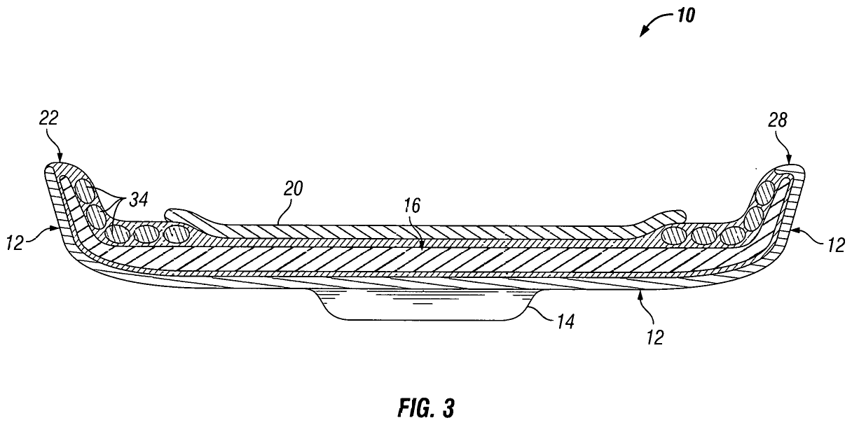

The sanitary pad 10 also includes an intermediate liquid absorbent member, generally designated, by the numeral, 16 which is adjacently coupled to and aligned with the base member 12. (FIG. 2). The intermediate liquid absorbent member 16, may be, for example a cotton like absorbent material. The intermediate liquid absorbent member 16 has also formed on an upper surface 18 thereof a pair of longitudinally extending raised absorbent guide members 20 (FIG. 3).

A first absorbent end member, generally designed, by the numeral, 22 is aligned with and coupled to extend outwardly from one end 24 and sides 26 of the intermediate liquid absorbent member 16. The first absorbent member 22 extends outwardly from the end 24 and sides 26 of the intermediate liquid absorbent member 16 in an oval configuration. A second absorbent end member, generally designated, by the numeral 28 is aligned with and coupled to extend outwardly from another end 30 and sides 32 of the intermediate absorbent member 16 in an oval configuration. The first and second absorbent members 22 and 28 are provided to eliminate leakage at the front and rear ends of the sanitary pad 10 particularly during periods of heavy flow. The first and second absorbent end members 22, and 28 can each be made, for example, of a single piece of cotton like absorbent material or a plurality of spaced aligned pieces of cotton like absorbent material 34 as illustrated in the figures.

When it is desired to use the sanitary pad 10 a user can simply couple the pad to the user's under garment by using the wing shaped engaging members 14 in a well known manner. This will properly position the intermediate liquid absorbent member 16 in the under garment of the user and allow the absorbent end member 22 to extend upwardly and outwardly in an oval configuration in the front of the user's torso and the absorbent end member 28 to extend upwardly and outwardly in an oval configuration in the rear of the user's torso. The longitudinally extending raised absorbent guide members 20 will then be positioned in the user's under garment to guide menstrual liquid overflow resulting from heavy flow towards the absorbent end member 22 and absorbent end member 28. As a result any leakage from the front and rear of the pad is contained.

This invention has been shown and described in what is considered to be the most practical and preferred embodiment. However, it should be recognized that changes may be made by those skilled in the art without departing from the spirit of the invention.

What is claimed:

1. A sanitary pad including:
a fluid-impervious base member;
a first engaging member formed on one side of the base member for couplingly engaging one side of an outside surface of an under garment of a user;
a second engaging member formed on another side of the base member for couplingly engaging another side of an outside surface of an under garment of a user;
an intermediate liquid absorbent member adjacently coupled to the base member;
a pair of spaced raised longitudinally extending upper absorbent members coupled to an upper surface of the intermediate liquid absorbent member;
a first liquid absorbent member coupled to extend outwardly from one end and sides of the intermediate liquid absorbent member; and
a second liquid absorbent member coupled to extend outwardly from another end and sides of the intermediate liquid absorbent member.

2. A sanitary pad as defined in claim 1 wherein base member includes a fluid-imperious plastic material.

3. A sanitary pad as defined in claim 2 wherein the intermediate liquid absorbent member includes an absorbent thin cotton-like material.

4. A sanitary pad as defined in claim 3 wherein the first engaging member includes an adhesive member for couplingly engaging one side of the outside surface of the under garment of the user.

5. A sanitary pad as defined in claim 4 wherein the second engaging member includes an adhesive member for couplingly engaging another side of the outside surface of the under garment of the user.

6. A sanitary pad as defined in claim 5 wherein the first liquid absorbent member is coupled to the one end and sides of the intermediate liquid absorbent member to extend outwardly therefrom in an oval configuration.

7. A sanitary pad as defined in claim 6 wherein the first liquid absorbent member is made of a plurality of spaced aligned pieces of cotton like absorbent material.

8. A sanitary pad as defined in claim 7 wherein the second liquid absorbent member is coupled to the other end and sides of the intermediate liquid absorbent member to extend outwardly therefrom in an oval configuration.

9. A sanitary pad as defined in claim 8 wherein the second liquid absorbent member is made of a plurality of spaced aligned pieces of cotton like absorbent material.

10. A sanitary pad as defined in claim 6 wherein the first liquid absorbent member is made of a single piece of cotton like absorbent material.

11. A sanitary pad as defined in claim 10 wherein the second liquid absorbent member is made of a single piece of cotton like absorbent material.
